(19) United States
(12) Patent Application Publication
(10) Pub. No.: US 2026/0231991 A1
(43) Pub. Date: Aug. 13, 2026

(54) Food Processing Line and Method for Processing Foodstuff

(71) Applicant: GEA FOOD SOLUTIONS BAKEL B.V., Bakel (NL)

(72) Inventors: Harrie Van Beers, Budel (NL); Joost Van Erp, Nuenen (NL)

(21) Appl. No.: 19/154,516

(22) PCT Filed: Jan. 25, 2024
(86) PCT No.: PCT/EP2024/051775

§ 371 Date: Aug. 7, 2025

(30) Foreign Application Priority Data
Feb. 17, 2023 (EP) 23157313.0

(51) Int. Cl. A23L 5/10; A22C 17/00; A23B 4/03; A23B 4/044

(52) U.S. Cl. CPC A23L 5/17; A22C 17/0053; A22C 17/0073; A23B 4/03; A23B 4/044

(57) ABSTRACT

Food processing line that includes a first oven configured for drying and smoking foodstuff, at least one microwave sensor configured to sense microwave transmission data of the foodstuff, the at least one microwave sensor includes a microwave transmitter and a microwave receiver that is spaced from the microwave transmitter, the microwave receiver configured to receive a transmission signal transmitted by the microwave transmitter, a foodstuff thickness measurement device that includes a laser configured to measure a thickness of the foodstuff; and a control unit configured to receive the microwave transmission data from the at least one microwave sensor, to receive the measured thickness of the foodstuff, and to control the first oven based on the microwave transmission data received from the at least one microwave sensor and the measured thickness.

### CROSS REFERENCE TO RELATED APPLICATIONS

This application is a National Stage Entry of PCT/EP2024/051775 filed on Jan. 25, 2024, which claims priority to EP 23157313.0 filed on Feb. 17, 2023, the entireties of which are all hereby incorporated by reference herein for all purposes.

### FIELD

The present invention relates to the field of food processing, in particular in-line food processing. It finds particular application in processing of foodstuff like meat, for example bacon, poultry and fish.

The present invention concerns a food processing line comprising an oven configured for drying, and preferably additionally for smoking, foodstuff. Additionally, the invention concerns a method for processing foodstuff, in particular meat—for example bacon—poultry or fish, in a food processing line comprising an oven configured for drying, and preferably additionally for smoking, foodstuff.

### BACKGROUND

EP 3 641 549 B1 discloses an in-line process for operating an oven which is configured for drying and smoking foodstuff. Using the known in-line oven, the liquid content of the processed foodstuff may be reduced so as to control shelf life and/or food safety.

It has however been observed that the resulting liquid content varies for different types of foodstuff which are supplied to the oven. Also, if different qualities of the same type of foodstuff are subjected to drying and smoking in the oven, the results may vary.

### SUMMARY

Against this background, the problem to be solved is to better control the drying of foodstuff for varying types and/or qualities of foodstuff.

For solving the problem, the invention proposes a food processing line comprising
a first oven configured for drying, and preferably additionally for smoking, foodstuff, and
at least one microwave sensor configured to sense microwave transmission data of the foodstuff, the microwave sensor comprising a microwave transmitter and a microwave receiver that is spaced from the microwave transmitter, the microwave receiver further being configured to receive a transmission signal transmitted by the microwave transmitter, and
a control unit configured to receive the microwave transmission data from the microwave sensor and to control the first oven based on the microwave transmission data received from the microwave sensor.

According to the invention, at least one microwave sensor is provided that is configured to sense microwave transmission data of the foodstuff which is dried, and preferably additionally smoked, in the oven. This microwave transmission data may be indicative of the quality of the foodstuff under test, in particular indicative of the liquid content of the foodstuff. The microwave transmission data is received by the control unit which is able to control the oven based on the microwave transmission data. Thus, the invention allows to control the oven depending on the liquid content of the foodstuff under test. Thereby, adapting the drying, and preferably smoking, operation to varying types and/or qualities of foodstuff may be improved or the drying, and preferably smoking, process can be optimized based on trend results.

The oven is preferably an in-line oven which includes a conveying device for conveying the foodstuff through the oven. The conveying device may be configured for transporting the foodstuff from an inlet of the oven through one or more chambers of the oven to an outlet of the oven. The conveying device may include a conveyor belt, in particular an endless conveyor belt. The conveying device may include a linear transport path from the inlet to the outlet. Alternatively or additionally, the conveying device may include a transport path with one or more helical sections.

The oven may include two or more chambers. The chambers of the oven may be controlled independently. At least a first chamber of the oven is configured for drying the foodstuff. The first chamber may additionally be configured for smoking the foodstuff. A second chamber of the oven may be configured for either drying or smoking the foodstuff or for combined drying and smoking of the foodstuff. The chambers may be arranged in the oven such that the foodstuff is first transported through the first chamber and consequently through the second chamber.

According to an optional embodiment of the invention, the food processing line includes a second oven for smoking the foodstuff, the second oven being arranged downstream the first oven, wherein the control unit is further configured to control the second oven based on the microwave transmission data received from the microwave sensor.

The microwave transmitter preferably transmits a frequency signal or a narrow bandwidth signal. The single frequency may be a frequency in the range of 0.1 GHz to 10 THz, in particular 810 MHz or 960 MHz or 1.165 GHz. The microwave transmitter preferably works with a transmit power of less than 1 mW, preferably less than 0.5 mW, e.g., 0.1 mW.

According to a preferred embodiment of the invention, the microwave sensor includes a processing unit that is configured to analyze the amplitude of the transmission signal received by the microwave receiver. Analyzing the transmission signal, in particular comparing the amplitude of the signal transmitted by the microwave transmitter and the transmission signal received by the microwave receiver, may indicate an amount of energy absorbed by the foodstuff. It has been found that the amplitude of the transmission signal is well suited for indicating the liquid content of the foodstuff. The processing unit may include a processor, e.g., a programmable processor, and/or a vector network analyzer (VNA). The processing unit may receive the signal transmitted by the microwave transmitter and the transmission signal received by the microwave receiver.

In this context, it is preferred that the processing unit is further configured to analyze a phase of the transmission signal received by the microwave receiver, preferably a phase difference between the signal transmitted by the microwave transmitter and the transmission signal received by the microwave receiver. By analyzing both the amplitude and the phase of the transmission signal the reliability of determining the liquid content can be improved as compared to analyzing only the amplitude.

According to a preferred embodiment of the invention, the control unit is further configured to adjust
a temperature inside the first, and if present, the second oven based on the microwave transmission data received from the microwave sensor, and/or
a humidity inside the first, and if present, the second oven based on the microwave transmission data received from the microwave sensor, and/or
an air flow velocity inside the first, and if present, the second oven based on the microwave transmission data received from the microwave sensor, and/or
a smoke supply into the first, and if present, the second oven based on the microwave transmission data received from the microwave sensor, and/or
a transport velocity of the foodstuff inside the first, and if present, the second oven based on the microwave transmission data received from the microwave sensor.

Preferably, the foodstuff is situated between the microwave transmitter and the microwave receiver during sensing with the microwave sensor.

According to a preferred embodiment of the invention, the food processing line includes a transport apparatus configured to transport the foodstuff from and/or to the first oven and, if present, the second oven. The microwave sensor(s) is/are preferably arranged at a position along the transport apparatus so as to sense the foodstuff prior to entering the first, and if present, the second oven and/or after leaving the first, and if present, the second oven. The transport apparatus may be a conveyor, in particular a belt conveyor or a walking beam conveyor.

According to a preferred embodiment of the invention, the transport apparatus is further configured to transport the foodstuff through a gap between the microwave transmitter and the microwave receiver of the microwave sensor. For example, a part of the transport apparatus may be arranged within the gap between the microwave transmitter and the microwave receiver in order to transport the foodstuff through the gap. In this example the transport apparatus may include an essentially horizontal conveyor and the one of the microwave transmitter and the microwave receiver may be arranged above the conveyor whereas the other one of the microwave transmitter and the microwave receiver is arranged below the conveyor so as to form an essentially vertical transmission path. According to an alternative example, both the microwave transmitter and the microwave receiver may be arranged on the same side of the transport apparatus, e.g., on the same side of a conveyor, whereas the transport apparatus does not reach into the gap between the microwave transmitter and the microwave receiver. In this example the transport apparatus may include an essentially horizontal conveyor whereas the microwave transmitter and the microwave receiver form an essentially horizontal transmission path.

According to a preferred embodiment of the invention, the control unit is further configured to control the transport apparatus. In particular, the control unit may be configured to adjust a transport velocity and/or a step displacement of the transport apparatus.

According to a preferred embodiment of the invention, the microwave sensor is arranged upstream the first oven. Arranging the microwave sensor upstream of the first oven allows to sense microwave transmission data of the foodstuff before drying the foodstuff. The liquid content of the foodstuff prior to drying may be indicative of the amount of liquid to be removed from the foodstuff during drying on the first oven. Thus, measuring the liquid content before drying allows to control the drying in order to achieve a predetermined liquid content in the foodstuff after drying, e.g., a predetermined liquid content which is above a predetermined minimum liquid content and below a predetermined maximum liquid content.

According to an alternatively preferred embodiment of the invention, the microwave sensor is arranged downstream the first oven. By arrangement of the microwave sensor downstream the first oven, it is possible to sense microwave transmission data of the foodstuff after drying the foodstuff. The liquid content of the foodstuff after drying may be indicative of the amount of liquid removed by drying. Thus, it is possible to check if the liquid removal has been achieved in the first oven and it is possible to adapt operation of the first oven, i.e., of the same batch, based on this assessment.

According to a preferred embodiment of the invention, the food processing line comprises at least two microwave sensors, wherein a first microwave sensor is arranged upstream the first oven and a second microwave sensor is arranged downstream the first oven. This embodiment allows to both measure the liquid content of the foodstuff prior to drying and after drying.

In an embodiment wherein the food processing line includes a second oven for smoking foodstuff, the food processing line may optionally include an additional microwave sensor, which is arranged downstream the second oven. By arrangement of the additional microwave sensor downstream the second oven, it is possible to sense microwave transmission data of the foodstuff after smoking the foodstuff. The liquid content of the foodstuff after smoking may be indicative of the total amount of liquid removed by drying and smoking. Thus, it is possible to check if the liquid removal has been achieved in the first and second ovens and it is possible to adapt operation of the first and/or second oven, i.e., of the same batch, based on this assessment.

According to a preferred embodiment of the invention, the food processing line comprises an injection apparatus configured to inject liquid into the foodstuff, wherein the injection apparatus is arranged upstream the first oven, and an additional microwave sensor which is arranged upstream the injection apparatus, wherein the control unit is further configured to receive additional microwave transmission data from the additional microwave sensor. The liquid may comprise salt and/or spices and/or oil and/or vinegar and/or phosphate and/or preservatives and/or flavours. The injection apparatus may include one or more injection needles coupled to a liquid reservoir. Arranging the additional microwave sensor upstream of the injection apparatus allows to sense microwave transmission data of the foodstuff before the liquid being injected into the foodstuff. The liquid content of the foodstuff prior to injection may be indicative of the maximum possible liquid uptake of the foodstuff supplied to the injection apparatus.

According to a preferred embodiment of the invention, the control unit may further be configured to control the injection apparatus and/or the first oven and/or, if present the second oven based on the additional microwave transmission data received from additional microwave sensor. The liquid to be injected using the injection apparatus may be a brine or a marinade. Thus, measuring the liquid content before liquid injection allows to control the liquid injection in order to achieve maximum liquid uptake in the foodstuff and/or to avoid injection of an amount of liquid which cannot be absorbed by the foodstuff. Optionally, the control unit may further be configured to control the injection apparatus based on the microwave transmission data received from the microwave sensor(s).

According to a preferred embodiment of the invention, the control unit is further configured to adjust
a liquid injection pressure based on the additional microwave transmission data received from the additional microwave sensor, and/or
a liquid injection volume based on the microwave additional transmission data received from the additional microwave sensor, and/or
a speed of an injection head of the injection apparatus based on the additional microwave transmission data received from the additional microwave sensor.

According to a preferred embodiment of the invention, the food processing line includes a foodstuff thickness measurement device, in particular comprising a laser or another height measurement device such as a mechanical probe, configured to measure the thickness of the foodstuff, preferably when being situated between the microwave transmitter and the microwave receiver. The control unit is preferably configured to additionally receive the measured thickness of the foodstuff and to control the oven and/or injection apparatus additionally based on the thickness. In particular, the microwave transmission data may be scaled by the measured thickness. The foodstuff thickness measurement device may be configured to measure a distance between the measurement device and a surface of the foodstuff when the foodstuff is arranged in a known distance to the measurement device, e.g., on a transport apparatus.

According to a preferred embodiment of the invention, the food processing line includes a foodstuff temperature measurement device, in particular comprising an IR temperature sensor, configured to measure the temperature of the foodstuff, in particular the surface temperature of the foodstuff, preferably when being situated in between the microwave transmitter and the microwave receiver. The control unit is preferably configured to additionally receive the measured temperature of the foodstuff and to control the oven and/or injection apparatus additionally based on the temperature.

According to a preferred embodiment of the invention, the control unit is further configured to
determine a content of certain ingredients of the foodstuff, in particular a fat content of the foodstuff, and/or
log the microwave transmission data received from the microwave sensor, and/or
log data received from the foodstuff thickness measurement device, and/or
log data received from the foodstuff temperature measurement device.

Preferably, the microwave transmission data received from the microwave sensor can be used to determine the chemical ratio of elements. The determined fat content can, e.g., be used to check foodstuff quality and/or to adapt the drying process in the first oven and/or the injection process in the injection apparatus and/or the smoking process in the second oven to the foodstuff quality. Logging the above-mentioned data may provide improved insight into the quality of the foodstuff and/or determining a trend and/or storing the data for track and trace reasons. The logged data may optionally be used to optimize the processing, in particular in the first oven and/or, if present, second oven and/or the injection apparatus, either automatically or manually by an operator of the apparatus.

For solving the problem, the invention further proposes a method for processing foodstuff, in particular bacon, in a food processing line comprising a first oven, at least one microwave sensor and a control unit, wherein
the foodstuff is dried using the first oven, and preferably additionally smoked using the first oven,
microwave transmission data of the foodstuff is sensed using the microwave sensor, wherein the microwave sensor comprises a microwave transmitter and a microwave receiver that is spaced from the microwave transmitter, wherein the microwave receiver receives a transmission signal transmitted by the microwave transmitter, and
drying the foodstuff using the first oven, and preferably additionally smoking the foodstuff using the first oven, is controlled by a control unit which receives the microwave transmission data from the microwave sensor based on the microwave transmission data received from the microwave sensor.

The method may achieve the same technical effects and advantages as described in conjunction with the inventive food processing line.

According to an optional embodiment of the invention, the food processing line includes a second oven, the second oven being arranged downstream the first oven, wherein the foodstuff is smoked using the second oven and smoking the foodstuff using the second oven is controlled by the control unit which receives the microwave transmission data from the microwave sensor based on the microwave transmission data received from the microwave sensor.

According to a preferred embodiment of the invention, the control unit adjusts
a temperature inside the first, and if present, the second oven based on the microwave transmission data received from the microwave sensor, and/or
a humidity inside the first, and if present, the second oven based on the microwave transmission data received from the microwave sensor, and/or
an air flow velocity inside the first, and if present, the second oven based on the microwave transmission data received from the microwave sensor, and/or
a smoke supply into the first, and if present, the second oven based on the microwave transmission data received from the microwave sensor, and/or
a transport velocity of the foodstuff inside the first, and if present, the second oven based on the microwave transmission data received from the microwave sensor.

According to a preferred embodiment of the invention, the foodstuff is transported from and/or to the first, and if present, the second oven by a transport apparatus, in particular a conveyor, of the food processing line, preferably wherein the transport apparatus transports the foodstuff through a gap between the microwave transmitter and the microwave receiver of the microwave sensor.

According to a preferred embodiment of the invention, the microwave sensor is arranged upstream the first oven and the microwave transmission data of the foodstuff is sensed before the foodstuff is dried in the oven.
According to an alternatively preferred embodiment of the invention, the microwave sensor is arranged downstream the first oven and the microwave transmission data of the foodstuff is sensed after the foodstuff has been dried in the oven.

According to a preferred embodiment of the invention, the food processing line comprises at least two microwave sensors, wherein a first microwave sensor is arranged upstream the first oven and senses first microwave transmission data of the foodstuff before the foodstuff is dried in the first oven, wherein a second microwave sensor is arranged downstream the first oven and senses second microwave transmission data of the foodstuff after the foodstuff has been dried in the first oven.

In an embodiment wherein the food processing line includes a second oven for smoking foodstuff, the food processing line may optionally include an additional microwave sensor, which is arranged downstream the second oven.

According to a preferred embodiment of the invention, the food processing line comprises
an injection apparatus which is arranged upstream the first oven, wherein liquid is injected into the foodstuff using the injection apparatus, and
an additional microwave sensor which is arranged upstream the injection apparatus and senses addition microwave transmission data of the foodstuff before liquid is injected in to the foodstuff,
wherein the control unit further receives additional microwave transmission data from the additional microwave sensor.

Preferably, injecting liquid into the foodstuff using the injection apparatus and/or drying the foodstuff using the first oven is further controlled by the control unit based on the additional microwave transmission data received from the additional microwave sensor.

According to a preferred embodiment of the invention, a foodstuff thickness measurement device, in particular comprising a laser or another heigh measurement device such as a mechanical probe, measures the thickness of the foodstuff, preferably when being situated in between the microwave transmitter and the microwave receiver

According to a preferred embodiment of the invention, a foodstuff temperature measurement device, in particular comprising an IR temperature sensor, measures the temperature of the foodstuff, in particular the surface temperature of the foodstuff, preferably when being situated in between the microwave transmitter and the microwave receiver.

According to a preferred embodiment of the invention, the control unit further
determines a content of certain ingredients of the foodstuff, in particular a fat content of the foodstuff, and/or
logs the microwave transmission data received from the microwave sensor, and/or
logs data received from the foodstuff thickness measurement device, and/or
logs data received from the foodstuff temperature measurement device.

Preferably, the microwave transmission data received from the microwave sensor can be used to determine the chemical ratio of elements. The determined fat content can, e.g., be used to check foodstuff quality and/or to adapt the drying process in the oven and/or injection process in the injection apparatus to the foodstuff quality. Logging the above-mentioned data may provide improved insight into the quality of the foodstuff and/or determining a trend and/or storing the data for track and trace reasons. The logged data may optionally be used to optimize the processing, in particular in the oven and/or in the injection apparatus, either automatically or manually by an operator.

Alternatively, or additionally, the beneficial features and/or advantageous embodiments described in conjunction with the food processing line may be implemented in the method for processing foodstuff according to the invention.

These and other characteristics, features and advantages of the present invention will become apparent from the following detailed description, taken in conjunction with the accompanying figures, which illustrates, by way of example, the principles of the invention. The description is given for the sake of example only, without limiting the scope of the invention.

### BRIEF DESCRIPTION OF THE FIGURES

FIG. 1 is a schematic representation of a food processing line in accordance with a first embodiment of the invention;

FIG. 2 is a schematic representation of a food processing line in accordance with a second embodiment of the invention;

FIG. 3 is a schematic representation of a food processing line in accordance with a third embodiment of the invention;

FIG. 4 is a schematic representation of a food processing line in accordance with a fourth embodiment of the invention;

FIG. 5 is a schematic representation of a microwave sensor in accordance with a first embodiment; and

FIG. 6 is a schematic representation of a microwave sensor in accordance with a second embodiment.

### DETAILED DESCRIPTION

The present invention will be described with respect to embodiments and with reference to the figures, but the invention is not limited thereto but only by the claims. The figures described are only schematic and is non-limiting. In the figures, the size of some of the elements may be exaggerated and not drawn on scale for illustrative purposes.

FIG. 1 illustrates a food processing line 10 in accordance with a first embodiment of the invention. The food processing line 10 includes a first oven 2 configured for drying foodstuff and a control unit 4 connected to the first oven 2 and configured to control the injection apparatus 2. The food processing line further includes a first transport apparatus 5 for transporting foodstuff to the first oven 2 and a second transport apparatus 5′ for transporting foodstuff from the first oven 2. The transport apparatuses 5, 5′ transport the foodstuff in a transport direction T. The transport apparatuses 5, 5′ may include a conveyor, e.g., a belt conveyor or a walking beam conveyor.

The oven is configured as an in-line oven and includes an internal conveying device for transporting the foodstuff inside the oven. The oven is preferably configured to additionally smoke the foodstuff after and/or during drying.

The food processing line 10 further comprises a microwave sensor 1 that is arranged upstream the first oven 2 in the transport direction T of the foodstuff. The microwave sensor 1 is configured to sense microwave transmission data of the foodstuff. The microwave sensor 1 comprises a microwave transmitter and a microwave receiver that is spaced from the microwave transmitter, the microwave receiver further being configured to receive a transmission signal transmitted by the microwave transmitter. The sensed microwave transmission data is sent to the control unit 4 which control unit receives the microwave transmission data from the microwave sensor and to controls the first oven 2 based on the microwave transmission data received from the microwave sensor 1. For example, the control unit 4 may be configured to adjust a temperature inside the oven 2 based on the microwave transmission data received from the microwave sensor 1, and/or a humidity inside the first oven 2 based on the microwave transmission data received from the microwave sensor 1, and/or an air flow velocity inside the first oven 2 based on the microwave transmission data received from the microwave sensor 1, and/or a smoke supply into the first oven 2 based on the microwave transmission data received from the microwave sensor 1, and/or a transport velocity of the foodstuff inside the first oven 2 based on the microwave transmission data received from the microwave sensor 1.

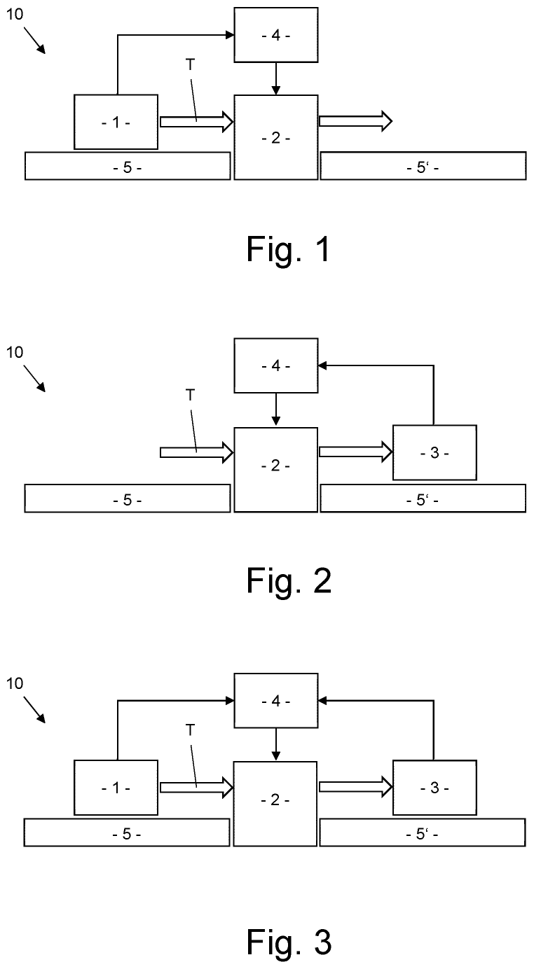

FIG. 2 illustrates a food processing line 10 in accordance with a second embodiment of the invention. The second embodiment is similar to the first embodiment. Thus, identical reference numerals are used for elements of identical function and reference is made to the description of the first embodiment. In contrast to the first embodiment, the food processing line of 10 of the second embodiment includes a microwave sensor 3 which is arranged downstream the first oven 2 in the transport direction T of the foodstuff. The microwave sensor 3 The comprises a microwave transmitter and a microwave receiver that is spaced from the microwave transmitter, the microwave receiver further being configured to receive a transmission signal transmitted by the microwave transmitter. The sensed microwave transmission data is sent to the control unit 4 which control unit receives the microwave transmission data from the microwave sensor and to controls the first oven 2 based on the microwave transmission data received from the microwave sensor 3. For example, the control unit 4 may be configured to adjust a temperature inside the first oven 2 based on the microwave transmission data received from the microwave sensor 3, and/or a humidity inside the first oven 2 based on the microwave transmission data received from the microwave sensor 3, and/or an air flow velocity inside the first oven 2 based on the microwave transmission data received from the microwave sensor 3, and/or a smoke supply into the first oven 2 based on the microwave transmission data received from the microwave sensor 3, and/or a transport velocity of the foodstuff inside the first oven 2 based on the microwave transmission data received from the microwave sensor 3.

FIG. 3 illustrates a food processing line 10 in accordance with a third embodiment of the invention. The third embodiment includes both a first microwave sensor 1 upstream the first oven 2 and a second microwave sensor 3 downstream the first oven 2. The third embodiment combines the aspects of the first and second embodiments. Thus, identical reference numerals are used for elements of identical function and reference is made to the description of the first and second embodiments.

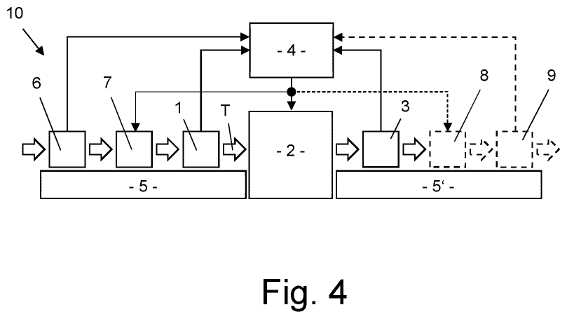

FIG. 4 illustrates a food processing line 10 in accordance with a third embodiment of the invention. The fourth embodiment includes all elements of the third embodiment. Thus, identical reference numerals are used for elements of identical function and reference is made to the description of the third embodiment. Further, the food processing line 10 includes an injection apparatus 7 upstream the first microwave sensor 1 and an additional microwave sensor 6 upstream the injection apparatus 7. The control unit 4 is further configured to control the injection apparatus 7 and the first oven 2 based on the additional microwave transmission data received from the additional microwave sensor 6. Optionally, the injection apparatus 7 may be controlled by the control unit 4 based on the microwave transmission data received from the first and second microwave sensor 1, 3.

Optionally, the food processing line 10 according to the fourth embodiment includes a second oven 8 downstream the second microwave sensor 3 and another additional microwave sensor 9 downstream the second oven 8. The second oven is configured for smoking the foodstuff. In this optional embodiment, the control unit 4 is configured to control the first oven 2 based on additional microwave transmission data received from the another additional microwave sensor 9. Optionally, the injection apparatus 7 and/or the second oven 8 may be controlled by the control unit 4 based on the microwave transmission data received from the first and second microwave sensor 1, 3.

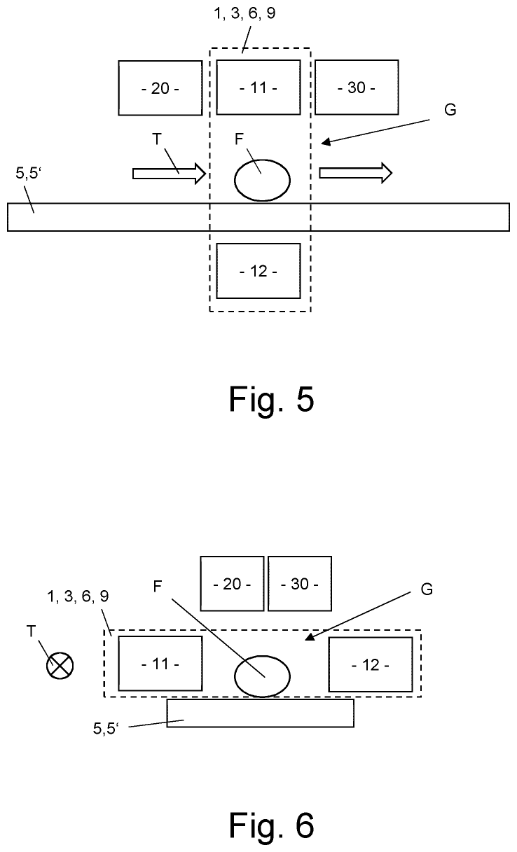

FIG. 5 illustrates a first embodiment of a microwave sensor 1, 3, 6, 9 which can be used on of the embodiments described before. The microwave sensor 1, 3, 6, 9 is arranged at a position along the transport apparatus 5, 5′ of the food production line 10. The transport apparatus 5, 5′ may be conveyor and is configured to transport the foodstuff F through a gap G between the microwave transmitter 11 and the microwave receiver 12 of the microwave sensor 1, 3. According to the first embodiment the transport direction T the foodstuff F is essentially horizontal. The microwave sensor 1, 3, 6, 9 is configured to comprise a transmission path which is essentially vertical. The microwave transmitter 11 is arranged above the transport apparatus 5, 5′ and the microwave receiver 12 is arranged below the transport device 5, 5′.

In the region of the microwave sensor 1, 3, 6, 9 also a foodstuff thickness measurement device 20 and a foodstuff temperature measurement device 30 are disposed. Both the foodstuff thickness measurement device 20 and a foodstuff temperature measurement device 30 are configured to measure the respective properties of the foodstuff F when being situated between the microwave transmitter 11 and microwave receiver 12.

The control unit 4 of the food processing line 10 the microwave sensor 1, 3, 6, 9 is used in, is preferably configured to additionally receive the measured thickness of the foodstuff F and to control the first oven 2 additionally based on the thickness. Further, the control unit 4 is preferably configured to additionally receive the measured temperature of the foodstuff F and to control the first oven 2 additionally based on the temperature.

FIG. 6 illustrates a second embodiment of a microwave sensor 1, 3, 6, 9 which can be used on of the embodiments of a food production line described before. The second embodiment of the microwave sensor 1, 3, 6, 9 is similar to the first embodiment as depicted in FIG. 5. Thus, identical reference numerals are used for elements of identical function and reference is made to the description of the first embodiment. In contrast to the first embodiment, the transmission path of the microwave sensor 1, 3, 6, 9 is oriented horizontally. Here, both the microwave transmitter 11 and the microwave receiver 12 are arranged above the transport apparatus 5, 5′. The microwave transmitter 11 and the microwave receiver 12 are separated by a gap G, wherein the transport apparatus 5, 5′ is configured to transport the foodstuff F through the gap G.

As already described in conjunction with FIG. 4, the second embodiment includes a foodstuff (layer) thickness measurement device 20 and a foodstuff temperature measurement device 30 configured to measure the respective properties of the foodstuff F when being situated in the gap G between the microwave transmitter 11 and microwave receiver 12.

### LIST OF REFERENCE SIGNS:

1 microwave sensor
2 oven
3 microwave sensor
4 control unit
5,5′ transport apparatus
6 microwave sensor
7 injection apparatus
8 oven
9 microwave sensor
10 food processing line
11 microwave transmitter
12 microwave receiver
20 foodstuff thickness measurement device
30 foodstuff temperature measurement device
F foodstuff
T transport direction

1. Food processing line comprising:
a first oven configured for drying and smoking foodstuff,
at least one microwave sensor configured to sense microwave transmission data of the foodstuff, the at least one microwave sensor comprising a microwave transmitter and a microwave receiver that is spaced from the microwave transmitter, the microwave receiver configured to receive a transmission signal transmitted by the microwave transmitter,
a foodstuff thickness measurement device comprising a laser configured to measure a thickness of the foodstuff; and
a control unit configured to receive the microwave transmission data from the at least one microwave sensor, to receive the measured thickness of the foodstuff, and to control the first oven based on the microwave transmission data received from the at least one microwave sensor and the measured thickness.

2. Food processing line according to claim 1, comprising a second oven configured for smoking the foodstuff, the second oven is arranged downstream from the first oven, wherein the control unit is further configured to control the second oven based on the microwave transmission data received from the at least one microwave sensor.

3. Food processing line according to claim 1, wherein the control unit is further configured to adjust:
a temperature inside of the first oven, and if present, inside of a second oven based on the microwave transmission data received from the at least one microwave sensor, and/or
a humidity inside of the first oven, and if present, inside of the the second oven based on the microwave transmission data received from the at least one microwave sensor, and/or
an air flow velocity inside of the first oven, and if present, inside of the second oven based on the microwave transmission data received from the at least one microwave sensor, and/or
a smoke supply into the first oven, and if present, into the second oven based on the microwave transmission data received from the at least one microwave sensor, and/or
a transport velocity of the foodstuff inside of the first oven, and if present, inside of the second oven based on the microwave transmission data received from the at least one microwave sensor.

4. Food processing line according to claim 1, wherein a transport apparatus is configured to transport the foodstuff from and/or to the first oven and, if present, from and/or to a second oven, the transport apparatus is further configured to transport the foodstuff through a gap between the microwave transmitter and the microwave receiver of the at least one microwave sensor, and the control unit is further configured to control the transport apparatus.

5. (canceled)

6. (canceled)

7. Food processing line according to claim 1, wherein the at least one microwave sensor is arranged upstream from the first oven.

8. Food processing line according to claim 1, wherein the at least one microwave sensor is arranged downstream from the first oven.

9. Food processing line according to claim 1, wherein the food processing line comprises at least two microwave sensors, wherein a first microwave sensor is arranged upstream from the first oven and a second microwave sensor is arranged downstream from the first oven.

10. Food processing line according to claim 7, wherein the food processing line comprises:
an injection apparatus configured to inject liquid into the foodstuff, wherein the injection apparatus is arranged upstream from the first oven, and
an additional microwave sensor which is arranged upstream from the injection apparatus,
wherein the control unit is further configured to receive additional microwave transmission data from the additional microwave sensor,
wherein the control unit is further configured to control the injection apparatus and/or the first oven and/or, if present, a second oven based on the additional microwave transmission data received from the additional microwave sensor.

11. (canceled)

12. (canceled)

13. Food processing line according to claim 1, comprising a foodstuff temperature measurement device, configured to measure a temperature of the foodstuff when being situated in between the microwave transmitter and the microwave receiver.

14. Food processing line according to claim 1, wherein the control unit is further configured to:
determine a content of certain ingredients of the foodstuff, and/or
log the microwave transmission data received from the at least one microwave sensor, and/or
log data received from the foodstuff thickness measurement device, and/or
log data received from a foodstuff temperature measurement device.

15. Method for processing foodstuff in a food processing line comprising a first oven, at least one microwave sensor, and a control unit, wherein the method comprises:
drying the foodstuff using the first oven and smoking the foodstuff using the first oven,
sensing microwave transmission data of the foodstuff using the at least one microwave sensor, wherein the at least one microwave sensor comprises a microwave transmitter and a microwave receiver that is spaced from the microwave transmitter, wherein the microwave receiver receives a transmission signal transmitted by the microwave transmitter,
measuring a thickness of the foodstuff using a foodstuff thickness measurement device comprising a laser when the foodstuff is situated in between the microwave transmitter and the microwave receiver, and
wherein the drying and the smoking of the foodstuff using the first oven is controlled by a control unit which receives the microwave transmission data from the at least one microwave sensor based on the microwave transmission data received from the at least one microwave sensor and the measured thickness.

16. Method according to claim 15, wherein the food processing line includes a second oven, the second oven being arranged downstream from the first oven, wherein the method comprises smoking the foodstuff using the second oven, and the smoking using the second oven is controlled by the control unit which receives the microwave transmission data from the at least one microwave sensor based on the microwave transmission data received from the microwave sensor.

17. Method according to claim 15, wherein the control unit adjusts:
a temperature inside of the first oven, and if present, inside of a second oven based on the microwave transmission data received from the at least one microwave sensor, and/or
a humidity inside of the first oven, and if present, inside of the second oven based on the microwave transmission data received from the at least one microwave sensor, and/or
an air flow velocity inside of the first oven, and if present, inside of the second oven based on the microwave transmission data received from the at least one microwave sensor, and/or
a smoke supply into the first oven, and if present, into the second oven based on the microwave transmission data received from the at least one microwave sensor, and/or
a transport velocity of the foodstuff inside of the first oven, and if present, inside of the second oven based on the microwave transmission data received from the at least one microwave sensor.

18. Method according to claim 15, wherein the method comprises transporting the foodstuff from and/or to the first oven, and if present, from and/or to a second oven by a transport apparatus of the food processing line, wherein the transport apparatus transports the foodstuff through a gap between the microwave transmitter and the microwave receiver of the at least one microwave sensor.

19. Method according to claim 15, wherein the at least one microwave sensor is arranged upstream from the first oven, and the microwave transmission data of the foodstuff is sensed before the foodstuff is dried in the first oven.

20. Method according to claim 15, wherein the at least one microwave sensor is arranged downstream from the first oven, and the microwave transmission data of the foodstuff is sensed after the foodstuff has been dried in the first oven.

21. Method according to claim 15, wherein the food processing line comprises at least two microwave sensors, wherein a first microwave sensor is arranged upstream from the first oven and senses first microwave transmission data of the foodstuff before the foodstuff is dried in the first oven, wherein a second microwave sensor is arranged downstream from the first oven and senses second microwave transmission data of the foodstuff after the foodstuff has been dried in the first oven.

22. Method according to claim 15, wherein the food processing line comprises:
an injection apparatus which is arranged upstream from the first oven, the method comprises injecting liquid into the foodstuff using the injection apparatus, and
an additional microwave sensor which is arranged upstream from the injection apparatus, the method comprises sensing additional microwave transmission data of the foodstuff using additional microwave sensor before liquid is injected in to the foodstuff,
wherein the control unit further receives additional microwave transmission data from the additional microwave sensor,
wherein the injecting liquid into the foodstuff using the injection apparatus and/or the drying the foodstuff using the first over is further controlled by the control unit based on the microwave transmission data received from the additional microwave sensor.

23. (canceled)

24. (canceled)

25. Method according to claim 15, wherein the method comprises measuring a temperature of the foodstuff or a surface temperature of the foodstuff using a foodstuff temperature measurement device comprising an IR temperature sensor when the foodstuff is situated in between the microwave transmitter and the microwave receiver.

26. Method according to claim 15, wherein the control unit further:
determines a content of certain ingredients of the foodstuff comprising a fat content of the foodstuff, and/or
logs the microwave transmission data received from the at least one microwave sensor, and/or
logs data received from the foodstuff thickness measurement device, and/or
logs data received from a foodstuff temperature measurement device.
